(19) United States
(12) Patent Application Publication
(10) Pub. No.: US 2007/0148095 A1
(43) Pub. Date: Jun. 28, 2007

(54) Fluorescent magnetic nanoparticles with specific targeting functions

(75) Inventors: Ming-Yao Chen, Taipei City (TW); Chi-Min Chau, Taichung County (TW); Hsiang Yuan Huang, Kaohsiung City (TW); Cheng-Yi Chen, Taipei County (TW); Pei-Shin Jiang, Taichung (TW); Chin-I Lin, Tainan County (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 11/430,894

(22) Filed: May 10, 2006

(30) Foreign Application Priority Data
Dec. 23, 2005 (TW) 94146105

(51) Int. Cl. A61K 49/10; C07K 16/46

(52) U.S. Cl. 424/9.34; 424/9.6; 530/391.1; 530/400; 977/930

(57) ABSTRACT

Magnetic nanoparticles with fluorescent properties and specific targeting functions. The fluorescent magnetic nanoparticle includes a magnetic nanoparticle, a biocompatible polymer chemically modifying the magnetic nanoparticle, a fluorescent dye coupled to the biocompatible polymer, and a specific targeting agent coupled to the biocompatible polymer. The fluorescent and magnetic properties of the nanoparticles provide different types of signal sources and therefore, prompt imaging using different types of imaging techniques to reconfirm foci is feasible.

### BACKGROUND OF THE INVENTION

1. Field of the Invention

The present invention relates to magnetic nanoparticles, and in particular to magnetic nanoparticles with fluorescent properties and specific targeting functions.

2. Description of the Related Art

In the biotechnology field, magnetic nanoparticles are applicable in imaging, diagnosis, therapy, biomaterial separation and so on. They are used, for example, in imaging as a contrast agent or a tracer to enhance the imaging contrast or to trace the presence of a certain disease. Furthermore, magnetic nanoparticles are also applicable in drug delivery and cancer therapy.

Currently, a number of image analysis techniques such as Computer Topography (CT), Magnetic Resonance Imaging (MRI), and ultrasound (US) are applied in, disease diagnosis. A popular analysis technique of computer topography employs an X-ray to image, for example, a human body by X-ray diffraction of various tissues with various densities. In addition, a contrast agent may be added during analysis to enhance contrast among different tissues or organs. The radiation of X-rays, however, may bring undesired side effects, and thus Magnetic Resonance Imaging (MRI) has been provided as an alternative analysis technique.

Magnetic resonance imaging is capable of showing several different characteristics of tissues. The level of tissue magnetization at specific signal recording times during the MR imaging cycle generally determines the brightness of a particular tissue in the MRI images. Contrast is produced when tissues do not have the same level of magnetization.

MRI provides more precise physiological information than is currently accessible from other imaging methods such as Computer Topography (CT) and ultrasound (US). Typically, tumor characteristics are first gathered by different types of imaging techniques, and tumor foci are then determined by MRI.

Iron oxide particles have been used in clinics as a contrast agent for MRI. Iron oxide particles shorten the effective transverse relaxation time (T2) of tissues that take up these particles. Compared with another category of MRI contrast agent, represented by gadolinium diethyltriamine pentaacetic acid (Gd-DTPA), which primarily shortens longitudinal relaxation time (T1) resulting in intensity enhancement, iron oxide detection is more sensitive. Current commercial MRI contrast agents, however, have poor specificity, and their contrast enhancement could be improved.

Although a number of imaging methods are available, they use different types of contrast agents. For example, reconfirmation of tumor foci by NIR (near-infrared) imaging after MRI requires further use of fluorescent agents. As a result, additional preparation is necessary, and diagnosis information may lose reference value due to a significant time delay.

### BRIEF SUMMARY OF THE INVENTION

It is therefore an object of the invention to provide a multi-modality contrast agent with specificity for both magnetic and optical imaging.

To achieve the above object, a targeting agent is coupled to magnetic nanoparticles to provide a target-specific. MRI contrast agent, thus enhancing targeting efficiency. Furthermore, the magnetic nanoparticles are coupled to a fluorescent dye to function as a contrast agent for optical imaging such as NIR imaging. Accordingly, the multi-modality contrast agent of the invention includes a magnetic nanoparticle, a biocompatible polymer chemically modifying the magnetic nanoparticle, a fluorescent dye coupled to the biocompatible polymer, and a specific targeting agent coupled to the biocompatible polymer.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 is a schematic view showing the fluorescent magnetic nanoparticle with specific targeting functions of the invention; and

FIGS. 2-5 are TEM micrographs of cell lines Hff, KB, HeLa, and MDA-MB-231 of Example 6, respectively.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides fluorescent magnetic nanoparticles with specific targeting functions. Specific targeting enhances targeting efficiency and provides a high contrast image of foci. The fluorescent and magnetic properties of the nanoparticles provide different types of signal sources and therefore, prompt imaging using different types of imaging techniques to reconfirm foci is feasible.

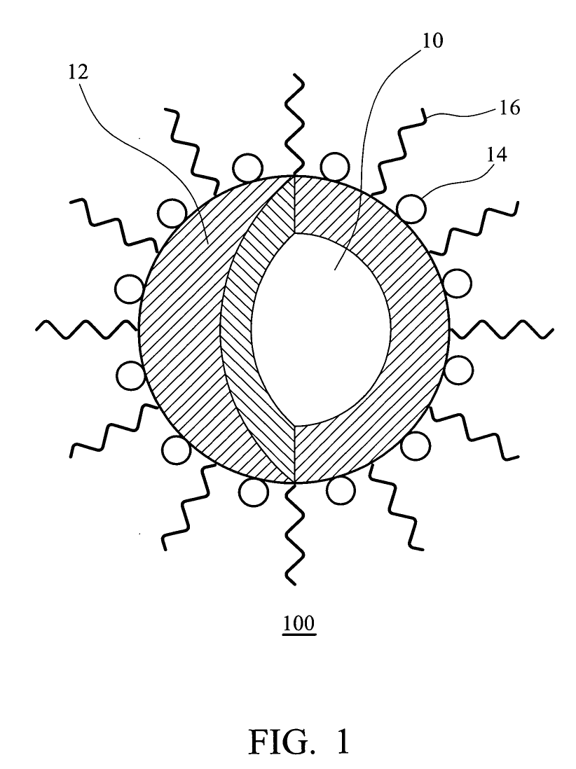

Referring to FIG. 1, the multi-modality magnetic nanoparticle 100 of the invention includes a biocompatible polymer. 12 chemically bonding to a magnetic nanoparticle 10. The biocompatible polymer 12 is coupled to a fluorescent dye 14 and a specific targeting agent 16. As shown in the figure, the biocompatible polymer 12 is preferably coated on the entire surface of the magnetic nanoparticle 10 to form a core-shell structure. More preferably, the biocompatible polymer 12 forms a monolayer coating on the magnetic nanoparticle 10.

The magnetic nanoparticle is preferably made of at least one of Fe, Co, Ni, and oxides thereof. It will be appreciated that the nanoparticle can be made of any single or composite magnetic material, although superparamagnetic materials are particularly preferred. A preferable diameter of the magnetic nanoparticle 10 is about 3-10 nm.

The biocompatible polymers 12 suitable for use in the invention include, but are not limited to, polyethylene glycol (PEG), polylactic acid (PLA), PLA-PEG, poly(glycolic acid) (PGA), poly(ε-caprolactone) (PCL), poly(methyl methacrylate) (PMMA), and the like. Chemical bonding between the biocompatible polymer 12 and the magnetic nanoparticle 10 can be established by reaction with a coupling agent (not shown). A preferable coupling agent is amino trialkoxysilane, such as 3-aminopropyltriethoxysilane (APS). The biocompatible polymer 12 provides water dispersity and blood compatibility for the magnetic nanoparticle 10 and simplify excretion from the host. It is noted that the biocompatible polymer 12 eliminates the need for using surfactant.

After the biocompatible polymer 12 is chemically bonded to the magnetic nanoparticle 10, its terminal groups are modified to allow bonding with the fluorescent dye 14 and the specific targeting agent 16. Those skilled in the art can attach any suitable targeting agents on the nanoparticle to give specificity thereto. Commonly used targeting agents include an antibody, a protein, a peptide, an enzyme, a carbohydrate, a glycoprotein, a nucleotide, and a lipid. For example, folic acid can be used to specify breast cancer cells with folate receptor. The structure of folic acid allows coupling with amine-terminated biocompatible polymer 12 by forming —CONH— linkage.

A fluorescent dye 14 is further coupled to the magnetic nanoparticle to provide optical signal for optical imaging techniques such as NIR imaging. Preferably, the fluorescent dye 14 is coupled to the biocompatible polymer 12 via covalent bonds. Suitable fluorescent dyes include organic or inorganic dyes and organometallic complexes. The excitation and emission wavelengths of the fluorescent dye may be ultraviolet (UV), near-infrared (NIR), or visible (VIS) light. The magnetic nanoparticle coupled with the targeting agent and fluorescent dye preferably has a diameter of about 15-100 nm. If the particle is too large, it may not be internalized into cells, or it can be captured by white blood cells through phagocytosis.

As described earlier, by coupling a fluorescent dye to magnetic nanoparticles, the fluorescent-magnetic nanoparticles can serve as a contrast agent for optical imaging as well as MRI, thus allowing easy confirmation of foci by different imaging techniques. Experimental study shows that coupling of the fluorescent dye does not decrease contrast enhancement of magnetic nanoparticles during MRI.

Without intending to limit it in any manner, the invention is further illustrated by the following examples.

### EXAMPLE 1

Nanoparticle Preparation

2.98245 g (0.015 mole) of FeCl₂.4H₂O was added to 8.109 g (0.03 mole) of FeCl₃.6H₂O and stirred to dissolve. The mixture was placed in a 2-necked flask and stirred at 500 rpm at 60° C., and a 5N NaOH solution was added at a rate of 100-150 μl/sec until black color appeared and a pH value of 13 was measured. After addition of NaOH, the reaction mixture was stirred for 15 minutes, cooled to room temperature, and centrifuged at 3000 rpm for 10 minutes to collect precipitates. The precipitates were re-dispersed in 0.5N HCl and centrifuged at 9000 rpm for 30 minutes to collect precipitates. The precipitates were washed with dimethyl sulfoxide (DMSO) and again, re-dispersed in DMSO and centrifuged at 9000 rpm for 30 minutes to collect supernatant. The supernatant was filtered through 0.1 μm polytetrafluoroethylene (PTFE) filter, and the resulting supernatant was collected as Fe₃O₄ nanoparticle suspension.

### EXAMPLE 2

Coupling with Biocompatible Polymer

0.167 mol of PEG biscarboxylate (Mn=600) and 0.48 mol of thionyl chloride were refluxed in a round bottom flask for 1.5 hours and distilled under reduced pressure (76 mmHg) for 1 hour. Thereafter, 0.44 mol of 2,2,2-trifluoroethanol was added to reflux and distilled under reduced pressure for 1 hour, giving PEG-ditrifluoroethylester.

135 mol of APS was added to PEG-ditrifluoroethylester for reaction for 8 hours. 0.866 mmol of iron oxide of Example 1 was dissolved in 100 ml of DMSO, followed by addition of 1.44 mol PEG-trifluoroethylester silane. 0.016 mol of ethylene diamine was added to the soltition and vigorously stirred for 2 hours to obtain iron oxide nanoparticles modified by amine-terminated PEG.

### EXAMPLE 3

Coupling with Targeting Agent

1.7 mmol of folic acid was completed dissolved in DMSO under sonication. 0.76 mmol of N-hydroxysuccinimide (NHS) and 3.9 mmol of 1-ethyl-3-(3-(dimethylamino)propyl) carbodiimide were added to the DMSO solution, followed by sonication at 60° C. for 1 hour. The pH value of the solution was adjusted to 9, and 200 mg (0.58 mmol) of iron oxide nanoparticle of Example 2 was added for reaction for 8 hours, giving iron oxide nanoparticles modified by folic acid.

### EXAMPLE 4

Coupling with Fluorescent Dye

The modified nanoparticles (2 mg/ml) of Example 3 were dissolved in 10 ml of deionized water, followed by addition of 1 ml CypHer5E (NIR dye from Amersham Bioscience Co., 10⁻⁶ mol/ml). The mixture was stirred for 7 hours to obtain fluorescent magnetic nanoparticles with specificity.

### EXAMPLE 5

Contrast Enhancement Test

The fluorescent magnetic nanoparticles of Example 4 were studied for the contrast enhancing properties by 0.47T 20 MHz MQ 20 mini-spec from Bruker Corporation. The measured r2/r1 ratio was 12 (201/16.7), which is much higher than 6.04 of commercial product, RESOVIST® from Schering Corporation.

### EXAMPLE 7

Cell Specificity Test

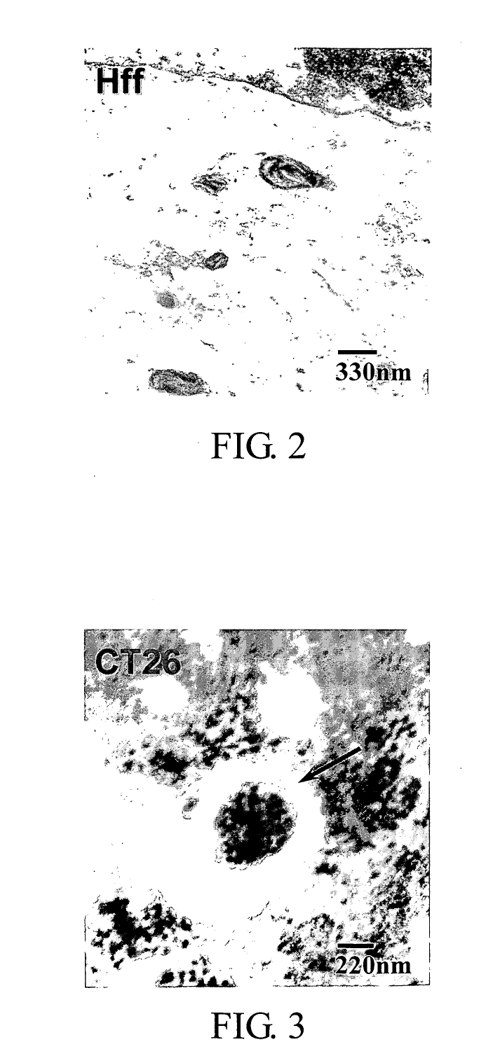

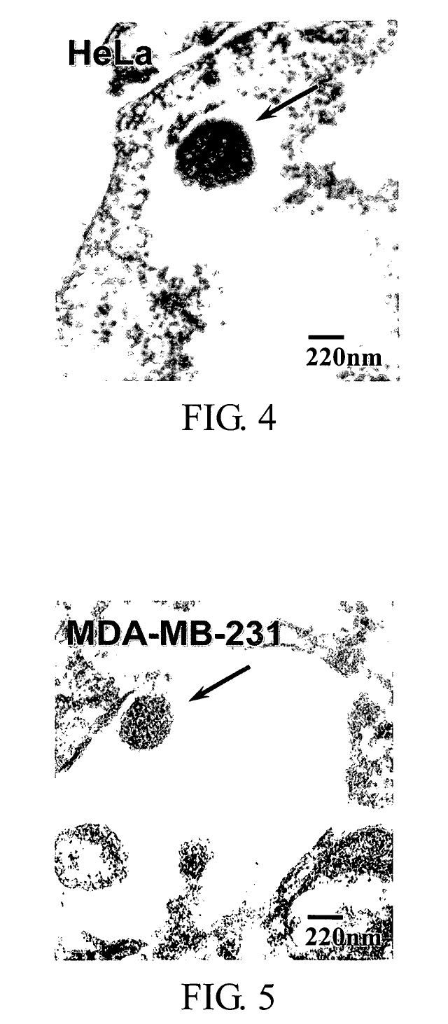

Cell lines of Hff (human foreskin fibroblast), HeLa (human epithelial carcinoma), KB (human nasopharynx carcinoma), and MDA-MB-231 (human breast cancer) were seeded respectively, followed by addition of 1 ml iron oxide nanoparticles (200 μg/ml) of Example 4. Each cell line was washed and harvested. Transmission electron microscopy (TEM) was used to confirm the internalization of iron oxide nanoparticles into cells. As shown in TEM micrographs of FIGS. 2-5, nanoparticles (as indicated by arrows in the figures) were internalized in those cells having folate receptor, i.e., KB, MDA-MB-231, and HeLa, while no internalization was observed in KB cells, which lacked folate receptor.

Flow cytometry was used to further quantify the nanoparticle uptake into each cells. The results are listed in the following table:

| Cell line | Uptake ratio of iron oxide nanoparticle |
|---|---|
| Hff (Control) | 4.95% |
| HeLa | 31.90% |
| MDA-MB-231 | 54.17% |
| KB | 70.26% |

KB, MDA-MB-231, and HeLA cells all showed considerable amounts of iron oxide uptake, compared with Hff cells that lacked folate receptor. This result conforms with the observation of TEM micrographs.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A fluorescent magnetic nanoparticle with specific targeting functions, comprising:
a magnetic nanoparticle;
a biocompatible polymer chemically modifying the magnetic nanoparticle;
a fluorescent dye coupled to the biocompatible polymer; and
a specific targeting agent coupled to the biocompatible polymer.

2. The fluorescent magnetic nanoparticle as claimed in claim 1, wherein the magnetic nanoparticle is a superparamagnetic nanoparticle.

3. The fluorescent magnetic nanoparticle as claimed in claim 1, wherein the magnetic nanoparticle comprises at least one of Fe, Co, Ni, and oxides thereof.

4. The fluorescent magnetic nanoparticle as claimed in claim 1, wherein the magnetic nanoparticle has a diameter of about 3-10 nm.

5. The fluorescent magnetic nanoparticle as claimed in claim 1, which has a diameter of about 15-100 nm.

6. The fluorescent magnetic nanoparticle as claimed in claim 1, wherein the fluorescent dye exhibits at least one of ultraviolet (UV), near-infrared (NIR), and visible (VIS) light excitation or emission wavelength.

7. The fluorescent magnetic nanoparticle as claimed in claim 1, wherein the fluorescent dye comprises an organic dye, an inorganic dye, or an organometallic complex.

8. The fluorescent magnetic nanoparticle as claimed in claim 1, wherein the specific targeting agent comprises an antibody, a protein, a peptide, an enzyme, a carbohydrate, a glycoprotein, a nucleotide or a lipid.

9. The fluorescent magnetic nanoparticle as claimed in claim 1, wherein the biocompatible polymer comprises at least one of polyethylene glycol (PEG), polylactic acid (PLA), PLA-PEG, poly(glycolic acid) (PGA), poly(ε-caprolactone) (PCL), and poly(methyl methacrylate) (PMMA).

10. The fluorescent magnetic nanoparticle as claimed in claim 1, wherein the biocompatible polymer comprises a terminal amino group.

11. The fluorescent magnetic nanoparticle as claimed in claim 1, wherein the fluorescent dye and the specific targeting agent are coupled to the biocompatible polymer by covalent bonds.

12. The fluorescent magnetic nanoparticle as claimed in claim 11, wherein the specific targeting agent and the biocompatible polymer are coupled by —CONH— linkage.

13. The fluorescent magnetic nanoparticle as claimed in claim 1, wherein the biocompatible polymer is coated on the magnetic nanoparticle to form a core/shell structure.

14. The fluorescent magnetic nanoparticle as claimed in claim 13, wherein the biocompatible polymer forms a monolayer coating on the magnetic nanoparticle.

15. The fluorescent magnetic nanoparticle as claimed in claim 1, wherein the biocompatible polymer is coupled to the magnetic nanoparticle by a coupling agent.

16. The fluorescent magnetic nanoparticle as claimed in claim 15, wherein the coupling agent is amino trialkoxysilane.
